(19) United States
(12) Patent Application Publication
(10) Pub. No.: US 2004/0087651 A1
(43) Pub. Date: May 6, 2004

(54) Monoterpene as a chemopreventive agent for regression of mammalian nervous system cell tumors, use of monoterpene for causing regression and inhibition of nervous system cell tumors, and method for administration of monoterpene perillyl alcohol

(76) Inventors: Clovis Orlando Pereira Da Fonseca, Rio de Janeiro (BR); Thereza Da Fonseca Quirico Dos Santos, Rio de Janeiro (BR); Maria da Gloria Da Costa Carvalho, Rio de Janeiro (BR); Gilda Alves Brown, Rio de Janeiro (BR)

Correspondence Address: JACOBSON HOLMAN PLLC, 400 SEVENTH STREET N.W., SUITE 600, WASHINGTON, DC, 20004, US

(21) Appl. No.: 10/318,385

(22) Filed: Dec. 13, 2002

(30) Foreign Application Priority Data
Dec. 17, 2001 (BR) 0107262-5

(51) Int. Cl. A61K 31/19; A61K 31/045

(52) U.S. Cl. 514/557; 514/729

(57) ABSTRACT

The present invention refers to a composition based on monoterpenes with chemopreventive and chemotherapeutic effects in malignant neoplasias of humans and animals containing from 0,03% to 30% of monoterpenes and 99,97% of solvents. Another objective of the present invention is an application of monoterpenes in inhibition of cell growth and metastasis control of primary tumors being applied in vitro and in vivo gliomas cell lines C6 and U 87 and A172. Further another objective of the present invention refers to a specific methods for applying the composition with chemopreventive and chemotherapeutic effects in humans and animals showing malignant neoplasias by inhalation and nebulization treatment, oral and intratumoral, followed or not by radiotherapy with dilutions from 0,03% to 30% of the monoterpene perillyl or its derived metabolites diluted in the solvents specified by the usual techniques.

### FIELD OF INVENTION

The present invention relates to treatment for mammalian nervous system cell tumors. In particular, the present invention relates to using of monoterpene perillyl alcohol to inhibit tumor formation and to cause tumor regression.

### BACKGROUND OF INVENTION

The progress in treating most common solid malignancies has slowed due at least in part to the lack of new effective agents. This has led to a new emphasis on cancer prevention and on developing therapeutic agents with novel mechanisms. Recent data suggest that monoterpenes, a class of non-toxic compounds that act through novel mechanism, may be useful for both chemoprevention and treatment of cancer.

Monoterpenoids, formed by the condensation of two isoprene molecules, are commonly and primarily produced by plants. They occur in monocyclic, bicyclic and acyclic forms in many commonly consumed fruits and vegetables, including citrus fruits and food flavoring such as mint, and are either simple or oxygenated hydrocarbons

These compounds have been shown to exert chemopreventive and chemotherapeutic activities in several tumor models and represent a new class of cancer therapeutic agents.

The perillyl alcohol (POH), also called p-metha, 1,7-diene-6-ol or 4-isopropenyl-cyclohexanecarbinol, is a monoterpene, and thus consists of two isoprene units manufactured by the mevalonate pathway. It is found in small concentrations in the essential oils of lavendin, pepermint, spearmint, cherries, celery seeds and several other plants. It has been shown to exert chemopreventive activity in mammary,(1) liver,(2) and prostatic (3) tumors models. It also has been used to treat a variety of rodent cancers, including pancreatic,(4) breast carcinomas (5) and melanoma.(6) In vitro data suggests that POH may be effective against neuroblastomas. (7) Drug-related activities that have been observed include the induction of apoptosis, cell cycle arrest, the inhibition of posttranslational modification of proteins that are involved in signal transduction, and differential gene regulation. Two mechanisms for POH activity has been proposed concerning Ras pathway: inhibition of Ras by farnesylation and a decrease in antigenic Ras protein levels. Our studies (8) evaluates the effects of the POH treatment of human glioma cell lines U 87 and A 172. Our preliminary in vitro studies have indicated that treatment of POH to a concentration of 30%, 3%, 0,3% and 0,03% inhibits the proliferation of glioma cell lines, and to a concentration of 0,3% producing changes in morphology, indicative of cellular differentiation, turning it a potential agent for treatment of patients with glioblastoma multiforme. In addition, the anti-metastatic activity of POH against these cells was evaluated using chick embryos as an in vivo model. The experiments have shown anti metastatic activity of POH when the U 87 and A 172 glial cells were exposed to a concentration of POH from 0,3% to 0,03% for 2 hours, prior to its inoculation in chick embryo chorioallantoic membrane model. This results showed the possible role of POH as in vivo anti-metastatic drug.

Although studies have shown that perillyl alcohol is not toxic to humans at the required doses levels, treatment with perillyl alcohol is not side-effects free, mainly gastrointestinal have been experienced when large doses are applied.

### SUMMARY OF THE INVENTION

The present invention provides a composition based on monoterpenes with chemopreventive and chemotherapeutic effects in malignant neoplasias of humans and animals, containing from 0.03% to 30% of monoterpenes and from 99,97% to 70% of solvents.

Further, the present invention relates to an application of monoterpenes in inhibition of cell growth and metastasis control of primary tumors, said monoterpenes being applied in vitro and in vivo gliomas cell lines C6 and U 87 and A172.

Another objective of the present invention are specific methods for applying the composition with chemopreventive and chemotherapeutic effects in humans and animals showing malignant neoplasias, by inhalation and nebulization treatment, oral and intratumoral, followed or not by radiotherapy, with dilutions from 0.03% to 30% of the monoterpene perillyl alcohol or its derived metabolites diluted in the solvents specified by the usual techniques.

The present invention provides methods for causing regression and inhibition of nervous system cell tumors in mammals. In one aspect, one administers to a nervous system cell tumor-containing mammal an effective amount of perillyl alcohol in concentrations ranging from 30% to 0,03%

The present invention is to cause regression of nervous system cell tumors by applying monoterpene perillyl alcohol in concentrations varying from 30% to 0,03%.

The present invention is to prevent a recurrence of nervous system cell tumors using monoterpene perillyl alcohol in patients that have already been treated for primary nervous system tumors and entered remission.

Other objectives, advantages and features of the present invention will become apparent upon examination of the specification, drawings and claims.

### BRIEF DESCRIPTION OF THE DRAWINGS

FIG. 1 shows a diagram of the chemical structure of monoterpenes.

FIG. 2 shows a diagram of the effect of perillyl alcohol on glial cell C6 growth.

FIG. 3 shows a diagram of the effect of perillyl alcohol on glial cell U87 growth.

FIG. 4 shows a diagram of the effect of perillyl alcohol on glial cell A 172 growth.

FIGS. 5, 6, 7, 8 and 9 shows a diagram of the effect of perillyl alcohol on morphology of glial cell A 172.

FIG. 10 shows a diagram of effect of perillyl alcohol on inhibition of protein expression of glial cell A 172.

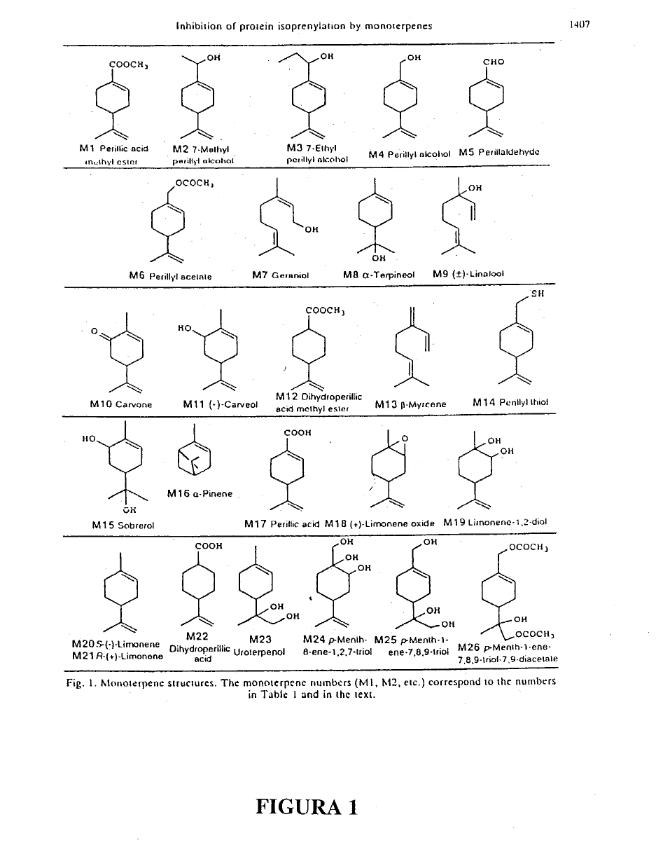

### DESCRIPTION OF THE PREFERRED EMBODMENTS

The present invention relates to a composition based on monoterpenes with chemopreventive and chemotherapeutic effects in malignant neoplasias of humans and animals, containing from 0.03% to 30% of monoterpenes and from 99,97% to 70% of solvents.

The solvents employed in this composition are solvents D_MEM, ethanol, bovine fetal serum. The monoterpene is the perillyl alcohol and its derived metabolites: dihidroperillyl acid and perillic acid. The composition contain 30% of monoterpenes, and said monoterpene is the perillyl alcohol at 0.3% of total composition. The perillyl alcohol correspond to be p-meta-1,7-dieno-6,1 or 4 isopropenylciclohexanecarbinol. This composition is applied in vivo and in vitro assays, and by application in glioma cell lines C6 and U 87 and A 172.

Another objective of this invention is the application of monoterpenes in inhibition of cell growth and metastasis control of primary tumors, the monoterpenes being applied in vitro and in vivo gliomas cell lines C6 and U 87 and A172, said monoterpenes being perillyl alcohol and its derived metabolites, said derived metabolites being perillic acid and dihidroperillic acid.

Another objective of the present invention are specific methods for applying the composition with chemopreventive and chemotherapeutic effects in humans and animals showing malignant neoplasias, by inhalation and nebulization treatment, oral and intratumoral, followed or not by radiotherapy, with dilutions from 0.03% to 30% of the monoterpene perillyl alcohol or its derived metabolites diluted in the solvents specified by the usual techniques.

The present invention also provides methods for causing regression and inhibition of nervous system cell tumors in mammals. In one aspect, one administers to a nervous system cell tumor-containing mammal an effective amount of perillyl alcohol in concentrations ranging from 30% to 0,03%

The present invention is to cause regression of nervous system cell tumors by applying monoterpene perillyl alcohol in concentrations varying from 30% to 0,03%.

The present invention is to prevent a recurrence of nervous system cell tumors using monoterpene perillyl alcohol in patients that have already been treated for primary nervous system tumors and entered remission.

### SPECIFIC EXAMPLES

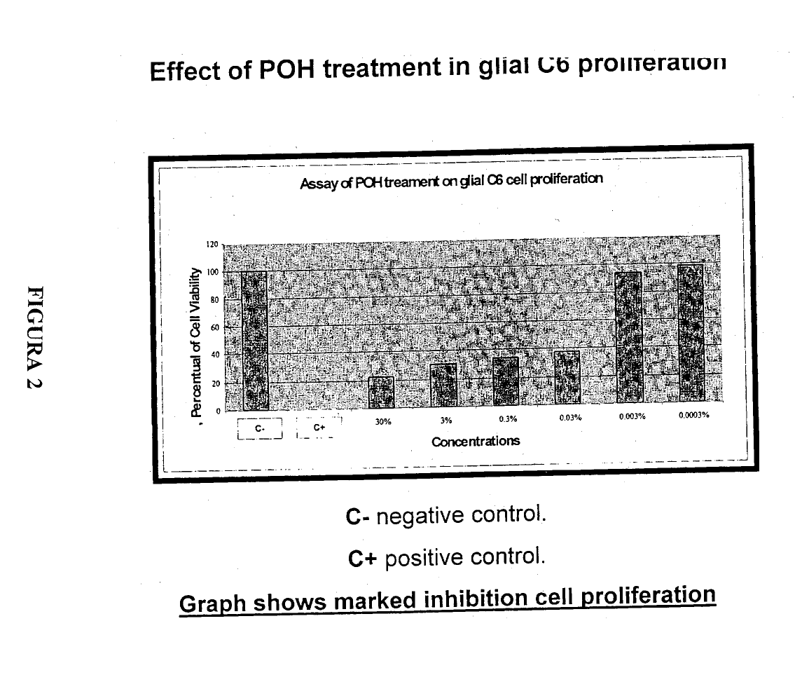

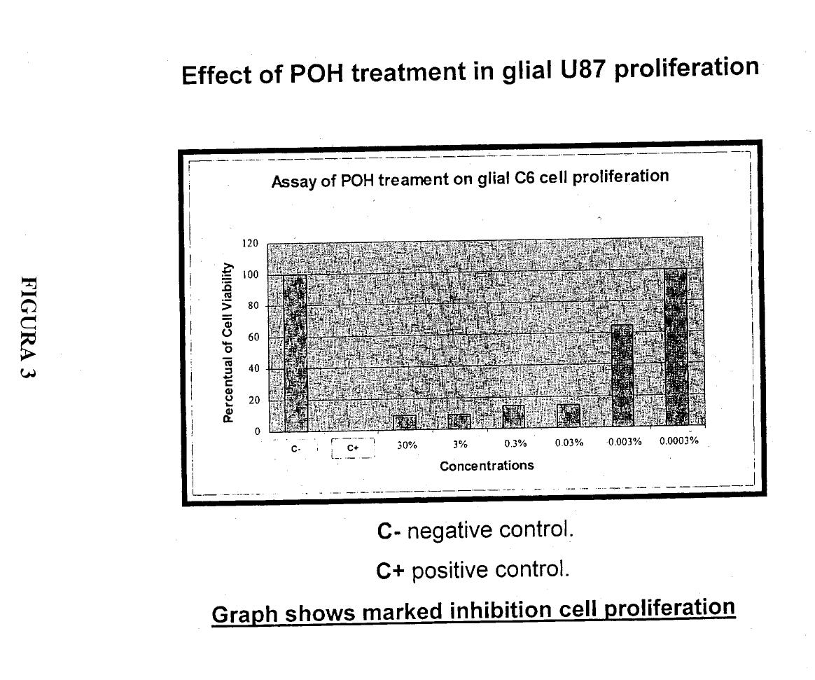

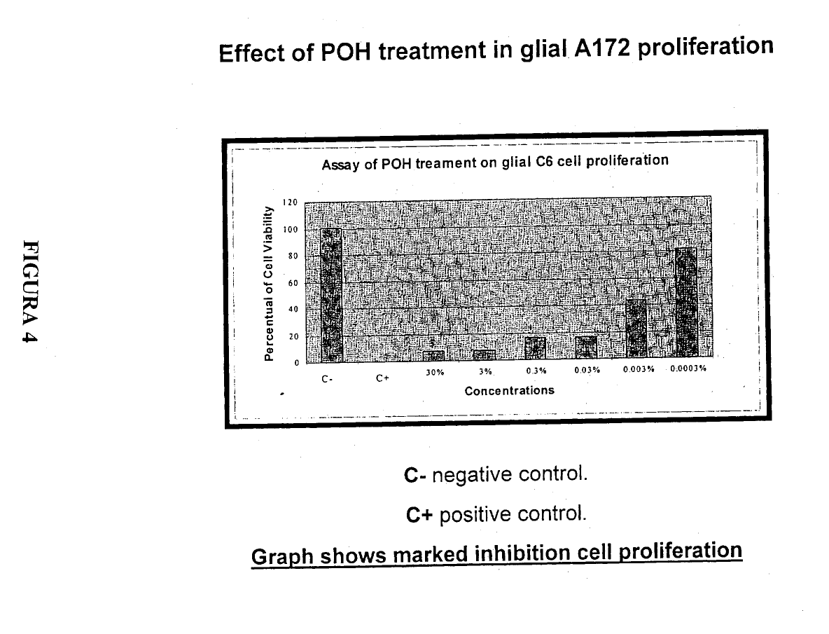

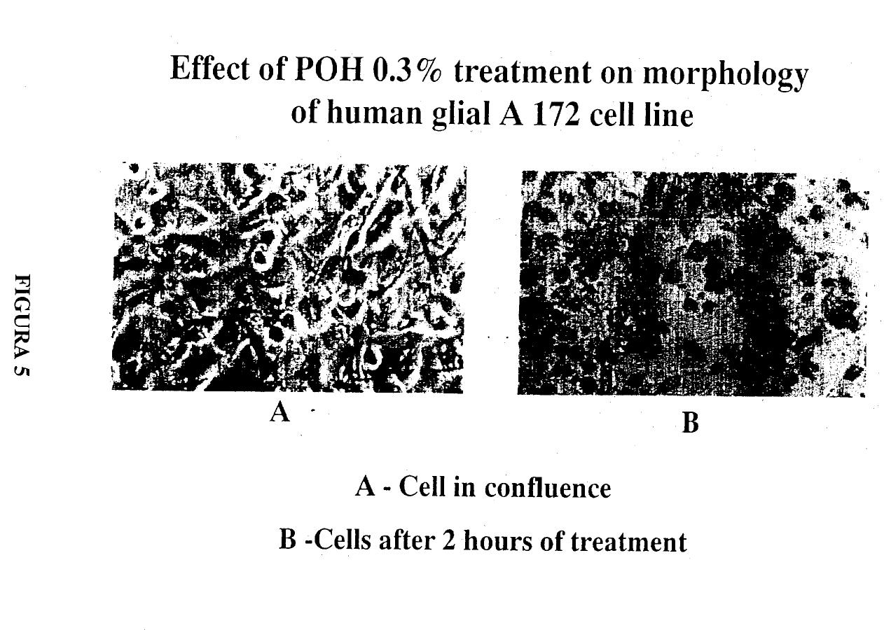

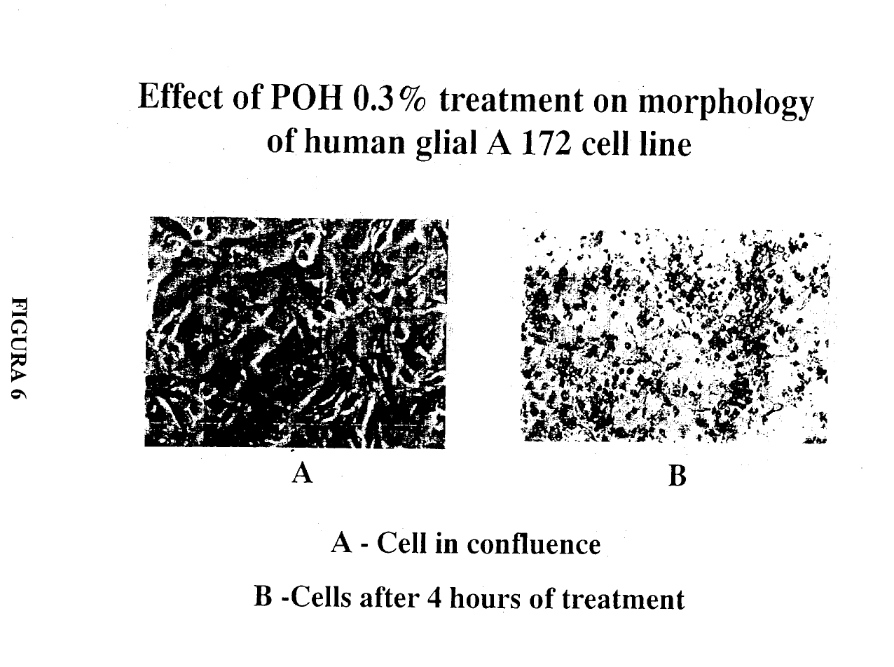

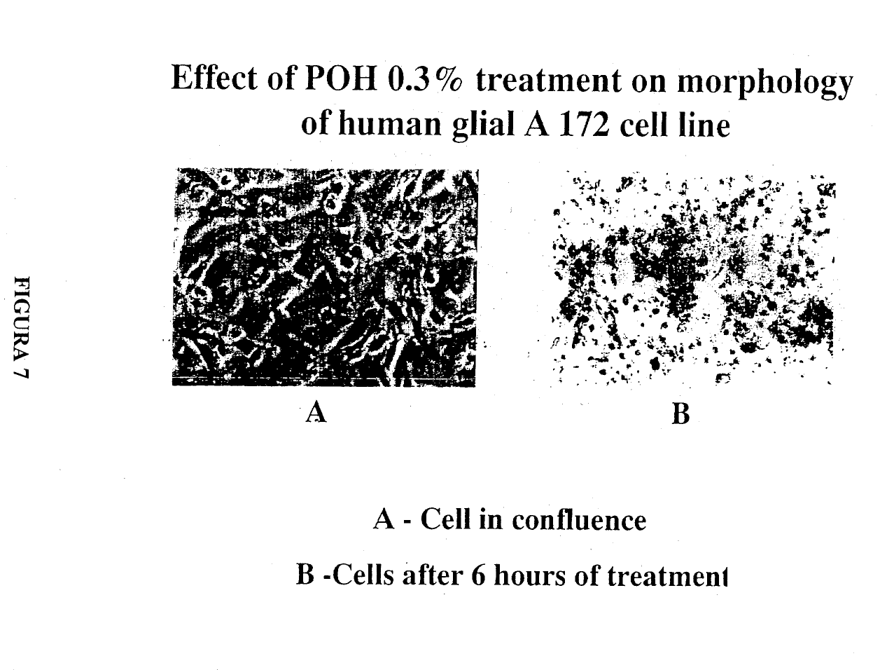

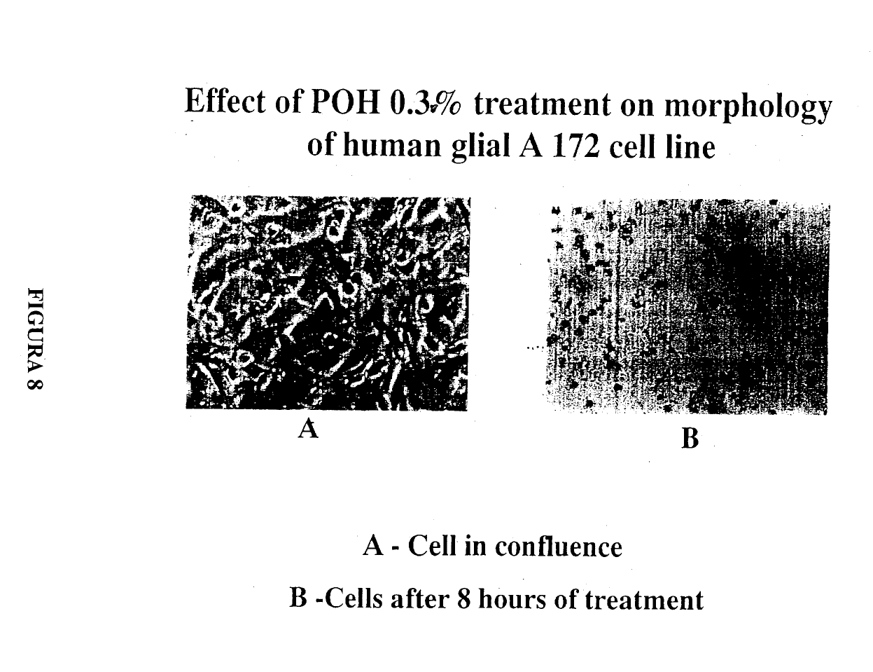

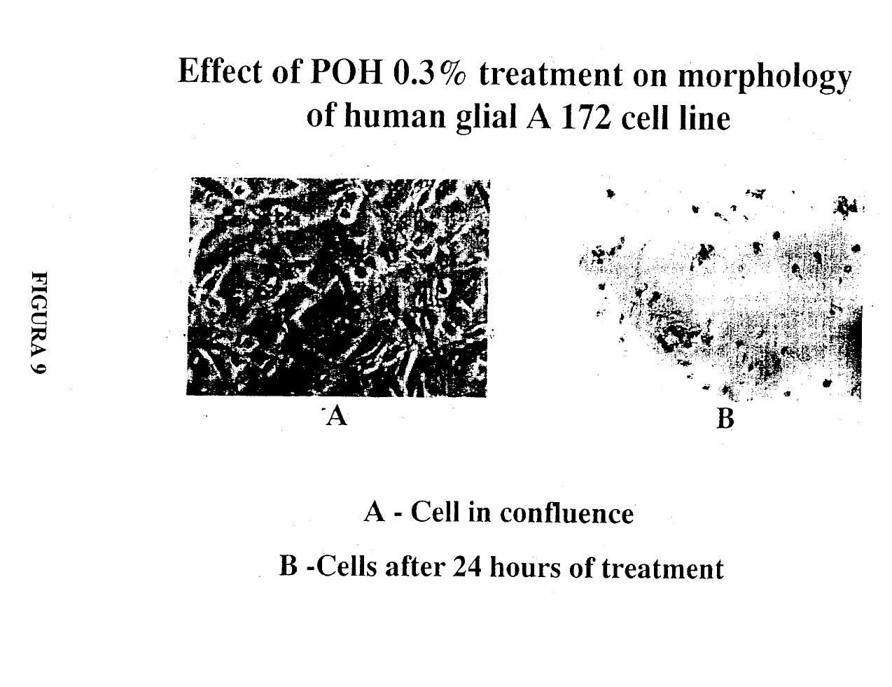

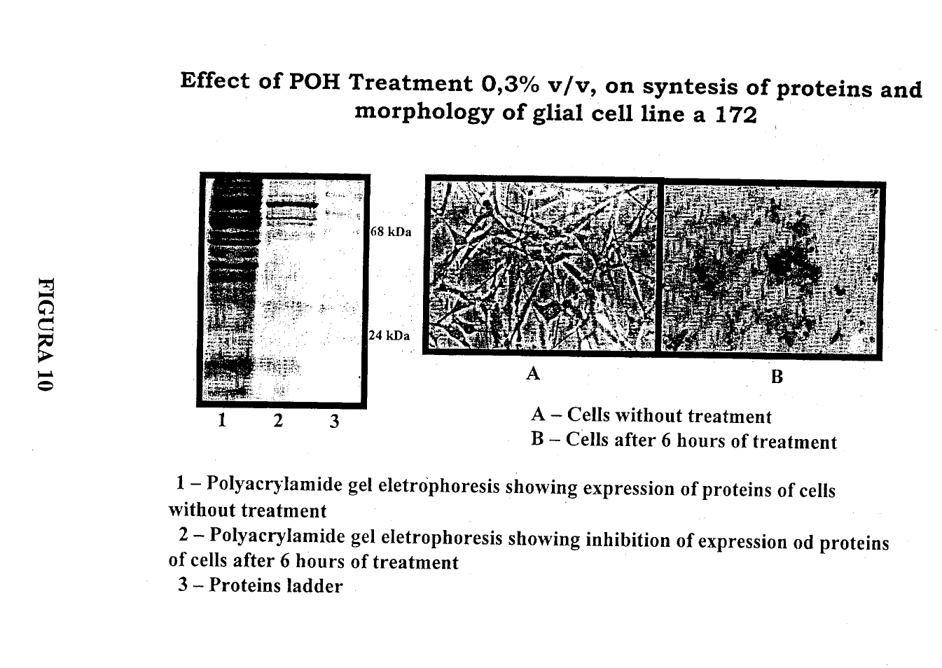

The current invention describes a method that causes inhibition or regression of nervous system cell tumors. By “nervous system cell tumors” we mean a condition in which mammalian patient has a malignant proliferation of nervous system cells. We mean “nervous system cell tumors” to be a synonymous with, glioblastoma multiforme, retinoblastoma, astrocitomas pylocitic astrocitomas grades II and III (anaplastic astrocitomas), meningeomas and brain metastatics tumors. Various concentrations of perillyl alcohol inhibited the growth of rats and two human glial cell line. FIGS. 2 to 4 correspond to a set of diagrams showing the effects of perillyl alcohol on different glial cell lines. Perillyl alcohol significantly inhibited cell growth in a dose dependent manner.

1—Biological activity

a—Cell Culture: Human malignant gliomas U 87 and A 172 cells were obtained from the American Type Culture Collection (ATCC), and grown as monolayers on the at 37° C., in Dulbecco's Modified Eagle Medium supplemented with 0.2 mM nonessential amino acids, 10% (v/v) fetal calf serum, penicillin (100 UI/mL), streptomycin (100 mg/mL) and amphotericin B (Fungizone, 2.5 mg/mL). For subcultures, confluent monolayers were gently washed with Dulbecco's phosphate—buffered saline (PBS, pH 7.2), and after a short exposure to trypsin, the cells were transferred to a new bottle containing complete growth media.

After determination of cell concentration applying a Neubauer Chamber, 2×106 cells were incubated for 2 hours at 37° C., in D-MEM medium containing concentrations varying from 0.3, 0.3 or 3% of perillyl alcohol (Sigma), prior to inoculation on CAM.

Cellular Proliferation Assay: the target cells (human glioma U 87 and A 172) were cultivated in plates of 96 wells at a concentration of 5.0×10⁵ cells by well. After the cellular adherence the cells were placed in multiple POH dilutions, in triplicates, having as positive control cells growing with detergent Tween 20. The negative control were cells growing without any additive. Concentrations varying from 30%, 3%, 0.03%, 0.003%, 0.0003% (v/v) were applied and after 24 hours of incubation 1.0 mg of hydrogen acceptor 3 (4,5 dimethylthiazol-2yi)-2-5 diphenyltetrazolium bromide (MTT) solution was added in each well, and the culture was incubated for additional 3 hours. The supernatant was removed from the plate well and the U87 and A 172 cells were calorimetrically revealed with the addition of 100 μl of DMSO in each well. An ELISA reader with 490 nm filter was used to read the results.

b—Chorioallantoic Membrane (CAM) Assay: seven days old fertilized specific pathogen free chick eggs were used in this study. By transillumination in a dark chamber, the egg air camera was marked on the eggshell. The following procedures were done under aseptic conditions, in the laminar chamber. An opening was made in the upper part and another in the lateral part of the eggshell, and the air camera was mechanically displaced to the upper side by applying negative pressure at the lateral opening. Both openings were sealed. In the upper part of the egg, a small window was made. On the upper CAM, the cells were inoculated, with a sterile pipette. Eggs 1, 2 and 3 received 2×106 cells previously treated with POH (0.03, 0.3 and 3%, respectively) diluted D-MEM medium. Egg 4 received 2×106 untreated cells in D-MEM medium. Egg 5 was used as negative control (no cells inoculated). The windows were sealed and the eggs were kept at 37° C. and 60% humidity, in the horizontal position, without agitation, for seven days

Opening of the eggs. Seven days after inoculation of the cells, a large window was opened in the eggshell, and 5 mL of the Carson's solution (17) were instilled below the CAM, and 1 mL on the membrane. After 3.5 hours at room temperature, the eggshell was cut around its circumference and the upper and lower CAMs were fractioned and conserved at 4° C. separately. The whole material from each was centrifuged for 10 minutes at 5800 g. The supernatant was discharged and 200 μL of PBS was added. This procedure was repeated twice, for a thorough wash of the material

DNA Extraction: the samples were macerated and 20 μL of the solution obtained were directly digested at 37° C. overnight in 100 μl of a solution with 100 mg/mL proteinase K as previously described Subsequently, DNA was extracted using a phenol: chloroform: isoamyl alcohol (25:24:1) pH 8.0 precipitated overnight in 2.5 volume of absolute ethanol at −20° C. and resuspended in 10 μL of TE buffer (10 mM Tris-HCl pH 8.0 and 1 mM EDTA)

DNA Amplification: 3 μL of each sample were submitted to PCR, in 50 μL of final reaction volume using Super Mix Gibco BRL reagent. 50 pmol of each primer with sense sequence 5′GGAATTCTGTTCACTTGTGCCCTGACTTTCAAC3′ and antisense sequence 5′GCAACCAGCCCTGTCGTGTCTCCA3′, flanking a region of exon 5 were used to amplify the p53 gene of the C6 cells. The PCR reaction was initiated by heating at 94° C. for 5 minutes to denature the DNA, followed by 35 reaction cycles of 1 minute at 94° C., 1 minute at 60° C. and 1 minute at 72° C. The PCR was completed with a final extension step of 15 minutes at 72° C. As positive control, a DNA preparation of A549 cells was used.

Electrophoresis: PCR products_ were submitted to electrophoresis in 8% polyacrylamide gel and then silver stained as previously described to allow visualization of the bands. Approximate fragments size was estimated by co-electrophoresing of a 100 Base Pair Ladder.

Studies of cell morphology:_A 172 and U 87 cells were cultivated in plates of 6 wells. After 24 hours, perillyl alcohol was added to a final concentration of 0.3% (v/v). Monolayers of cells treated with POH were photographed by an optic microscope inverted NIKON TMS, to register morphological changes induced by the treatment. All studies of cell morphology were performed at least three times to ensure accurate interpretation.

2—Use as a Chemotherapeutic

a—Other Tumor Types

Studies have demonstrates inhibition of cell growth by in a variety of human cell types: neuroblastoma, leukemia, colon, melanoma, pancreatic mammary and liver tumors. (33)

b—Appropriate Dose and Administration

Peryllyl alcohol is used of approximately 2.5 g/Kg mammal weight. While this dose might be useful for human treatment, a more conventional treatment dosage would be to calculate the dosage on a surface area basis. In mammalian in vivo experiment is used a surface area dose of approximately 10-15 g/day. Perillyl alcohol is administered the dose orally. However, other administration modes, such as intravenous administration, would also be appropriate. (33)

3—Toxicity

Toxicity was limited to weight loss in perillyl alcohol-fed rats. Studies shown that both perillyl alcohol and control mammals experienced initial weight loss followed by weight gain and a plateau. The perillyl alcohol-fed rats did not achieve weights similar to control. In toxicity studies 2,5% perillyl alcohol diet was the maximum dose tolerated by the animals. (33) Our studies in vivo shown that, after injection of 1 ul of perillyl alcohol into the brain of rats, has no toxicity in tissues of lung, liver, and brain of rats.

4—Inhibition of Protein Isoprenylation in M600B Human Cells.

The ability of monoterpenes to inhibit isoprenylation of proteins was tested in the immortalized human mammary epithelial cell line M600B. (18) As in the NIH3T3 cells, M600B cells treated with 5 mM of the monoterpene exhibited a marked decrease in the intensity of the 21-26 kDa bands. The effect was dose responsive, for significant inhibition of protein isoprenylation. (18) Our experiments also illustrated, in all cases we have examined POH treatment in glial cell lines, the ability of perillyl alcohol in inhibition protein expression.

### Publications

1—Haag J D, Gould M N. Mammary carcinoma regression induced by perillyl alcohol, a hydroxylated analog of limonene. Cancer Chemother Pharmacol 1994;34:477-483.

2—Mills J J, Chari R S, Boyer I J, et al. Induction of apoptosis in liver tumors by the monoterpene perillyl alcohol. Cancer Res 1995;55:979-983.

3—Jeffers L. The effect of perillyl alcohol on the prolifation of human prostatic cell lines. Proc Am Assoc Cancer Res 1995;36:303.

4—Stark J M, Burke Y D, McKinzie J H, et al. Chemotherapy of pancreatic cancer with the monoterpene perillyl alcohol. Cancer Lett 1995;96:15-21.

5—Lockwood K, Moesgaard S, Yamamoto T, Folker K. Progress on therapy of breast cancer with vitamin Q 10 and the regression of metastases. Biochem Biophys Res Commun 1995;212:172-177.

6—He L, MoH, Hadisusilo S, et al. Isoprenoids supress the growth of murine B 16 melanomas in vitro and in vivo. J Nutr 1997; 127:668-674

7—Shi W, Gould M N. Induction of differentiation in neuro-2 A cells by monoterpene perillyl alcohol. Cancer Lett 1995;95: 1-6.

8—Effects of perillyl alcohol in glial C6 cell line in vitro and anti-metastatic activity in chorloallantoic membrane model. Balassiano I. T, Silva N. H, Bozza M, Orlando da Fonseca C, Da Gloria Carvalho M. International Journal of Molecular Medicine.2002 Dez; 10(6): 785-788.

9—Elegbede, et al., “Inhibition of DMBA—induced Mammary Cancer By the Monoterpene D-Limonene,” carcinogenis, 5[5]:661, (1984).

10—Elegbede, et al., “Regression of Rat Primary Mammary Tumors Following Dietary d-Limonene,”JNCI, 76[2]:323-325 (1986).

11—Haag, et al., “Limonene-incuced Complete Regression of Rat Mammary Carcinomas,” Proc. Of the Amer. Assoc. For Cancer Research, 32:402 Abs #2391 (1991).

12—Elson, et al., “Anti-carcinogenic Activity of d-limonene During the initiation And Promotion/Progession Stages of DMBA-incuced Rat Mammary Carcinogenesis,” Carcinogenesis, 9[2]331:332 (1988).

13—Maltzaman, et al., “The Prevention of Nitrosomethylurea-in duced Mammary Tummors by d-Limonene and Orange Oil,”, Carcinogenesis, 10, [4]:781-783 (1989).

14—Van Duren, et al.,“Cocarcinogenic And Tumor-Promoting Agents In Tabacco Carcinogenesis,”Journ. Of the Natl. Cancer Institute, 56[6]:1237-1242 (1976).

15 -Homburger, et al., “Inhibition of Isoprenylation Of 21-26 KDa Proteins By The Anticarcinogen d-Limonene And Its Metabolites,” The Journ, of Biol. Chem., 266[26]:17679-17685 (September 1991).

16—Gould, “Chemoprevention And Treatment of Experimental Mammary Cancers By Limonene,” Proc. of the Amer. Assoc. For Cancer Res., 32:474-475 (1991).

17—Abtract 3134, Crowell, et al., “Human Metabolism of Orally Administered D-Limonene,” proc. Of the Amer. Assoc. For Cancer Research, 33:524 (1992) (see also 3135).

18—Crowell, et al., “Identification of Metabolites of the Antitumor Agent d-limonen Capable of Inhibiting Protein Isoprenylation and Cell Growth,” Cancer Chemotheraphy and Pharmacology, 31:205-212, 1992.

19—Crowell, et al., “Chemoprevention of Mammary Carcinogenesis by Hydroxylated Derivative of d-limonene,” Carcinogenesis, 13(7): 1261-1264 (1992).

20—Haag, et al., “Limonene-induced Regression of Mammary Carcinomas,” Cancer Research, 52:4021-4026 (1992).

21—Ren, et al., “Inihibition of Ubiquinone and Cholesterol Synthesis by the Monoterpene Perilly Alcohol,” cancer Letters, 76:185-190 (1994).

22—Ren, et al., “Inbition of Ubiquinome Biosynthesis by the Monoterpene Perilly Alcohol (POH),” Proc. Of the Amer. Assoc. For Cancer Research, 34:548 (1993).

23—Plenary Session 4: Mechanismos of action of chemopreventive agents: Basic science and clinical applications, Gould, et al., “Chemoprevention of mammary cancer by monoterpeners,” Proceedings, Eighty-Fourt annual Meeting, American Association for Cancer Research p. 572-73, vol. 34 (1993).

24—Gould, et al., Proposal, “Phase I Evaluation of Perillyl Alcohol (NSC 641066) in Patients With Cncer,” distributed in Aug., 1993.

25—Gould, et al., “Perillyl Alcohol(NSC (641066) and Limonene (NSC 844) Summary for the Decision Network,” distributed in February, 1994.

26—Crowell, et al., “Structure-Activity Relationships Among monoterpene Inhibitors of Protein Isoprenylation and Cell Proliferation,” Biochemical Pharmacology, 47:14-15, 1994.

27—Gould, et al., “Cellular and Molecular Aspects of the Multistage Progression of Mammary Carcinogenesislin Humans and Rats,” Cancer Biology, 4;161-169 (1993).

28—Clark et al., “Molecular Pathogenesis of Ph-Positive Leukemias,” Ann. Ver. Med., 40:113-22 (1989).

29—Abstract 3135, Haag, et al., Proceedings of the Eighty-Third Annual Meeting of the American Association for Cancer Research, vol. 33, p. 524, March 1992.

30—Ren, et all., “Inhibition of ubiquinone and cholsterol synthesis by the monoterpene perillyl alcohol,” Cancer Letters 2[3]:185-190,1994.

31—Bronfen, et al., “Inhibition of human pancreaatic carcinoma cell proliferation by perillyl alcohol,” proc. Annu. Meet. Am. Assoc. cancer Research 35:431,1994.

32—Crowell, et al., “Structure-activity relationsship among monoterpene inhibitions of protein isoprenylation and cell proliferation,”Biochem. Pharmacol. 47[8]:1405-1415,1994.

33—Perillyl Alcohol: Applications in Oncology James Belanguer, N. D Altern Med Ver 1998;3(6):448457

Primary Examiner—Jeromr d. Goldberg

Attorney, Agent, or Firm—Quarles & Brady

1. Composition based on monoterpenes with chemopreventive and chemotherapeutic effects in malignant neoplasias of humans and animals, characterized by containing from 0.03% to 30% of monoterpenes and from 99,97% to 70% of solvents.

2. Composition according to claim 1, characterized by applied solvents D_MEM, ethanol, bovine fetal serum.

3. Composition according to claim 1, characterized by the monoterpene be perillyl alcohol and its derived metabolites: dihidroperillyl acid and perillic acid.

4. Composition according to claim 1, characterized by containing 30% of monoterpenes.

5. Composition according to claim 1, characterized by the monoterpene be perillyl alcohol at 0.3% of total composition.

6. Composition according to claim 4, characterized by the perillyl alcohol correspond to be p-meta-1,7-dieno-6,1 or 4 isopropenylciclo-hexanecarbinol.

7. Composition according to claim 1, characterized by application in vivo and in vitro assays.

8. Composition according to claim 6, characterized by application in glioma cell lines C6 and U 87 and A 172.

9. Application of monoterpenes in inhibition of cell growth and metastasis control of primary tumors, characterized by being applied in vitro and in vivo gliomas cell lines C6 and U 87 and A172.

10. Application according to claim 9, characterized by monoterpene be perillyl alcohol and its derived metabolites.

11. Application according to claim 10, characterized by penillyl alcohol derived metabolites being perillic acid and dihidroperillic acid.

12. Method for applying the composition with chemopreventive and chemotherapeutic effects in humans and animals showing malignant neoplasias, characterized by inhalation and nebulization treatment, oral and intratumoral, followed or not by radiotherapy, with dilutions from 0.03% to 30% of the monoterpene perillyl alcohol or its derived metabolites diluted in the solvents specified by the usual techniques.

13. Method according to claim 12, characterized by concentration 0.3% to 30%.

14. Method for causing regression and inhibition of nervous system cell tumors in mammals, characterized by administrate to a nervous system cell tumor-containing mammal an effective amount of perillyl alcohol in concentrations ranging from 30% to 0,03%

15. Method to cause regression of nervous system cell tumors, characterized by applying monoterpene perillyl alcohol in concentrations varying from 30% to 0,03%.

16. Method to prevent a recurrence of nervous system cell tumors, characterized by using monoterpene perillyl alcohol in patients that have already been treated for primary nervous system tumors and entered remission.


SEQUENCE LISTING (2 sequences)

SEQ ID NO: 1 - DNA, 33 bp, Artificial Sequence
  Description of Artificial Sequence Sense sequence.
ggaattctgttcacttgtgccctgactttcaac

SEQ ID NO: 2 - DNA, 24 bp, Artificial Sequence
  Description of Artificial Sequence Antisense sequence.
gcaaccagccctgtcgtgtctcca